(19) United States
(12) Patent Application Publication
(10) Pub. No.: US 2022/0156810 A1
(43) Pub. Date: May 19, 2022

(54) METHOD AND SYSTEM TO DELIVER TIME-DRIVEN ACTIVITY-BASED-COSTING IN A HEALTHCARE SETTING IN AN EFFICIENT AND SCALABLE MANNER

(71) Applicant: KONINKLIJKE PHILIPS N.V., EINDHOVEN (NL)

(72) Inventors: Dieter Maria Alfons VAN DE CRAEN, Balen (BE); Christoph Tobias WIRTH, Vellmar (DE); Steffen Clarence PAUWS, Eindhoven (NL); Daniele DE MASSARI, Eindhoven (NL)

(21) Appl. No.: 17/441,472

(22) PCT Filed: Mar. 19, 2020
(86) PCT No.: PCT/EP2020/057650

§ 371 Date: Sep. 21, 2021

Related U.S. Application Data
(60) Provisional application No. 62/821,473, filed on Mar. 21, 2019.

(51) Int. Cl. G06Q 30/02; G16H 10/60

(52) U.S. Cl. CPC G06Q 30/0283; G16H 10/60

(57) ABSTRACT

A method for managing healthcare costs includes generating a first set of event logs based on first information, extracting traces from the first set of event logs for different episodes of care, generating a second set of event logs based on second information, determining a plurality of clusters based on the second set of event logs, grouping the traces to form sets of traces linked to the plurality of clusters, and inputting the sets of traces into a process mining module.

### TECHNICAL FIELD

This disclosure relates generally to processing information, and more specifically, but not exclusively, to managing the allocation and cost of providing healthcare resources.

### BACKGROUND

Healthcare organizations around the world are shifting towards value-based care in order to improve clinical outcomes relative to associated costs. However, these organizations have no way of accurately determining the actual costs associated with these outcomes. For example, existing methods use process mapping in away that is resource intensive and produces inaccurate results. These effects have been made worse by the high variation in processes used in the healthcare field, without any way of determining beforehand the actual costs associated with these processes. Shadowing, interviewing, and other techniques have been used in an attempt to mitigate these effects. However, they have proven ineffective because the sample size is very limited, e.g., sufficient sample size requires extensive time for observation. This leads to errors, inefficiencies, and a waste of resources.

As a further attempt of overcoming these drawbacks, automated techniques have been produced such as process mining. However, these methods are impractical because of the wide variety of care paths that are available for any given healthcare, and this is true even when the process is to be applied to a specific health condition. These attempts also offer no solution in providing an accurate determination of the actual costs associated with implementing various healthcare outcomes.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

In accordance with one or more embodiments, a method for managing healthcare costs includes generating a first set of event logs based on first information, extracting traces from the first set of event logs for different episodes of care, generating a second set of event logs based on second information, determining a plurality of clusters based on the second set of event logs, grouping the traces to form sets of traces linked to the plurality of clusters, and inputting the sets of traces into a process mining module.

The first information may include patient or medical-care information, the second information includes patient profile data, and each of the plurality of clusters may correspond to different groups of patients. The patient profile data may correspond to at least one of medical conditions, insurance participation, or treatment options. The method may include identifying patients corresponding to the patient profile data of the second set of event logs based on the traces extracted for the first set of event logs. Patients in each patient group may have at least one common feature.

The method may include generating, by the process mining module, a Markov chain for each cluster in the set of clusters, wherein a state space of the Markov chain of each cluster is given by different tasks indicated in the event logs of the first set. The method may include assigning a cost to states in one or more of the Markov chains, and calculating weights for the Markov chains based on a ratio of a number of the traces assigned to each of the Markov chains to a size of the trace set including the number of traces.

The method may include calculating distance measures for respective pairs of the Markov chains, wherein each of the distance measures provides an indication of similarity between the Markov chains in a respective one of the pairs. The method may include determining an expected cost of each of the different episodes of care for each patient group based on the calculated costs, weights, and distance measures. The method may include estimating profitability for each of the different episodes of care for each patient group, the profitability estimated based on a budget for each different episode contracted with a payer less the expected cost of the different episode.

In accordance with one or more embodiments, a system for managing healthcare costs includes an interface to receive first information and second information and a processor configured to generate a first set of event logs based on the first information, extract traces from the first set of event logs for different episodes of care, generate a second set of event logs based on the second information, determine a plurality of clusters based on the second set of event logs, group the traces to form sets of traces linked to the plurality of clusters, input the sets of traces into a process mining module, and output results based on the process mining module for display.

The first information may include patient or medical-care information, the second information may include patient profile data, and each of the plurality of clusters may correspond to different groups of patients. The patient profile data may correspond to at least one of medical conditions, insurance participation, or treatment options. The processor may be configured to identify patients corresponding to the patient profile data of the second set of event logs based on the traces extracted for the first set of event logs. Patients in each patient group may have at least one common feature.

The processor may be configured to generate a Markov chain for each cluster in the set of clusters, wherein a state space of the Markov chain of each cluster is given by different tasks indicated in the event logs of the first set. The processor may assign a cost to states in one or more of the Markov chains. The processor may calculate weights for the Markov chains based on a ratio of a number of the traces assigned to each of the Markov chains to a size of the trace set including the number of traces. The processor may calculate distance measures for respective pairs of the Markov chains, wherein each of the distance measures provides an indication of similarity between the Markov chains in a respective one of the pairs. The processor may determine an expected cost of each episode of care for each patient group based on the calculated costs, weights, and distance measures.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates an embodiment for managing healthcare resources and costs;

FIG. 2 illustrates an embodiment of a method for managing healthcare resources and costs;

FIG. 3 illustrates additional operations of the method in FIG. 2;

FIG. 4 illustrates an example of a Markov chain; and

FIG. 5 illustrates an embodiment of a system for managing healthcare resources and costs.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

Example embodiments describe a method and system for allocating costs and healthcare services to patients in an improved manner. In one embodiment, a costing model is used to assign cost rate and time spent to care delivery processes. These metrics are then used, for example, to provide an accurate estimate actual costs and a reliable measure of profitability for an episode budget contracted with a payer for delivering care for a defined patient pool. The method and system, therefore, includes what can be referred to as time-driven, activity-based-costing approach to control costs and allocate healthcare services in a manner more efficient and scalable than previous methods.

In one embodiment, the method and system may use process mining techniques to determine costs and healthcare service allocation. The process mining techniques may involve applying data mining algorithms to event log data in order to identify trends, patterns, and/or details contained in event logs. In one implementation, the method and system may create an event logs of traces that record services rendered to patients grouped on the basis of patient characteristics for a given medical condition before process mining. The resulting processes from the process mining may be more useful to investigate, as they relate to similar patients and as such are easier to compare.

In employing this approach, an underlying assumption may be that similar patients treated for the same medical condition will go through similar (or commensurable) care delivery processes. The process mining result for each patient sub-group may be represented as a Markov chain facilitating a costing method to assign cost rate and time spent to states within the Markov chain representing care delivery processes. The Markov chain may then be used to estimate profitability for an episode budget contracted with a payer for delivering care for a defined patient pool.

Through such embodiments, a thorough understanding of the real costs involved in the treatment of conditions may improve value-based care, both from the standpoints of the patient and the healthcare organization.

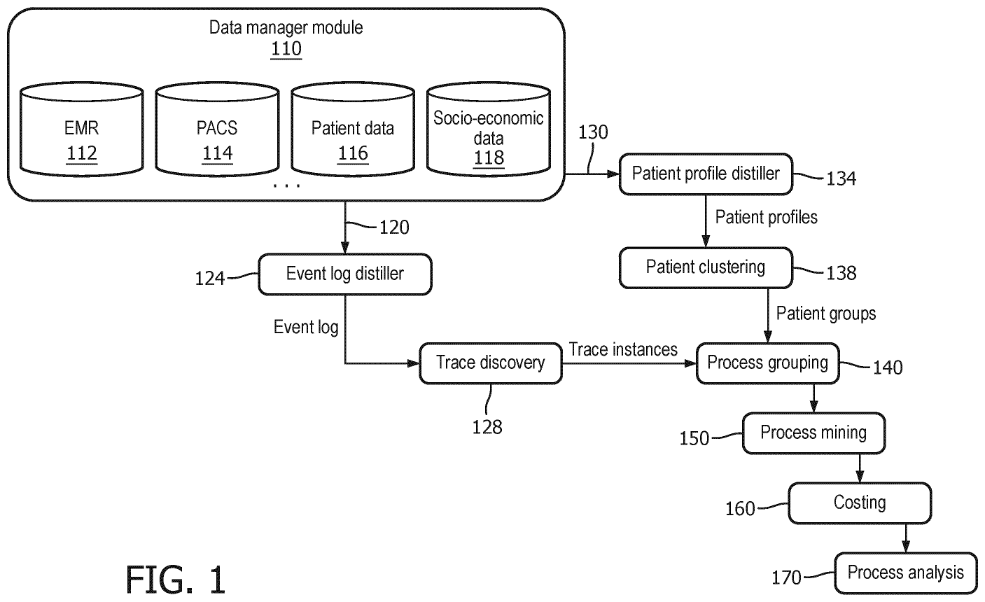

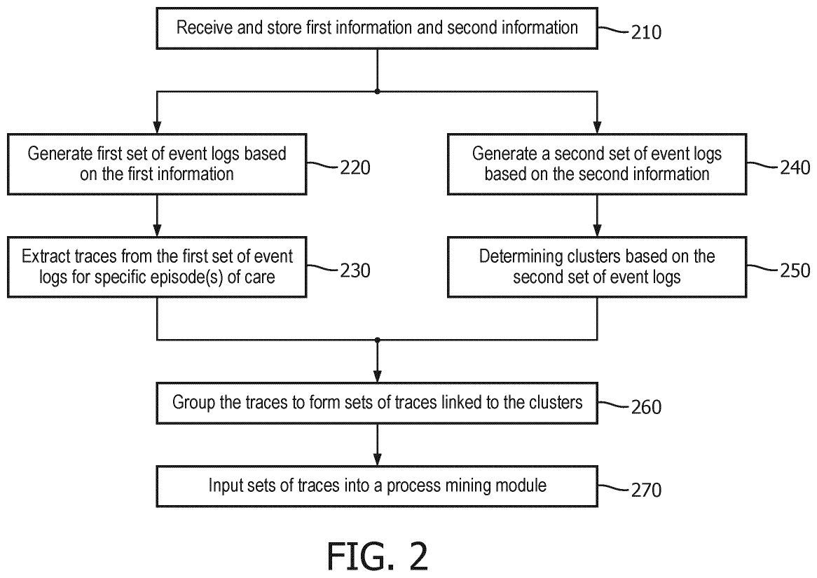

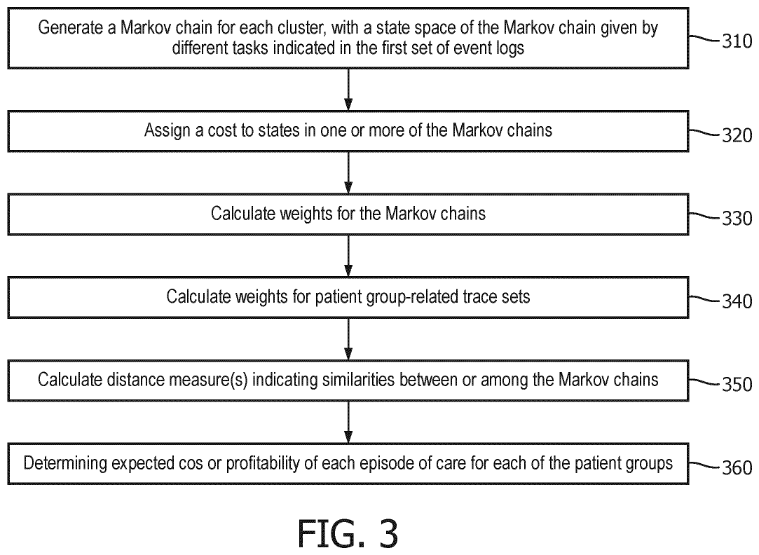

FIG. 1 illustrates an embodiment of a system 100 for managing the allocation of healthcare costs and services, and FIGS. 2 and 3 illustrate embodiments of a method including operations that may be performed, in entirely or partially, by the system of FIG. 1.

Referring to FIGS. 1, 2, and 3, the system 100 includes a number of modules that manage a variety of healthcare processes from a patient (or beneficiary) perspective. Using these modules, a pipeline is formed to obtain and pre-process patient, medical, and other related information. The pipeline is followed by a module for determining beneficiaries or patients eligible for healthcare process for a particular medical condition (e.g., episode of care). Another module or set of processing stages groups event logs based on patient (or beneficiary) characteristics. These pipelines and modules may be applied before process mining, which may allow process mining to be performed more accurately and efficiently.

For example, the operations performed by the modules and processing stages may allow similar patients to be grouped and more easily compared. This is because similar patients may be expected to be treated for the same medical condition using similar (or commensurable) care delivery processes. Due to the more homogenous nature of patients, the total variation within these processes may be lowered compared to the overall variation within all processes for the condition.

Referring to FIG. 1, this system 100 includes a data manager module 110 which flows into a first processing path 120 and a second processing path 130. The data manager module 110 receives, at operation 210, information from one or more data sources to be used in generating patient profiles and event logs. Some or all of the information from the data sources may be received, for example, from a cloud-based network. In these or other embodiments, the data sources may be stored in a blockchain or another decentralized database managed within a distributed network. The connection to the data manager module 110 may be pushed from the data sources and/or may be accessed by the data ingestion module 110 according to a predetermined time schedule or whenever requested.

Once received, the information may be placed in one or more storage areas of the data manager module 110, either as received and/or in pre-processed form. The storage areas may store, for example, various forms of electronic medical records (EMRs) 112, picture archiving and communication system (PACs) imaging and reports 114, patient data 116 such as clinical status and demographics information, and socio-economic information 118. The data manager module 110 may also store computerized physician order entry (CPOE) data, test and lab results, and/or other information indicative of other system or healthcare diagnostics and treatments relating to utilization of medical devices, order entries, and lab requests. The information stored in the data manager module 110 may be used to generate one or more event logs.

The first processing path 120 includes an event log distiller 124 and a trace discovery stage 128. The event log distiller 124 generates, at operation 220, event logs based on first information stored in the data manager module 110. The event log distiller may perform this operation by gathering event data and other information relating to patients and beneficiaries from the data manager module 110, and then extracting the first information from the event data. The first information may include, for example, tasks that have been executed and rendered to the patient, the order of execution of these tasks, the resources used to perform the tasks, and the process instance to which the tasks belong. The event logs and associated information may allow process mining to be performed in a subsequent operation. Before being used in process mining, the extracted information may be converted to a format compatible with a process mining solution, e.g., XES (eXtensible Event Stream) or MXML (Mining eXtensible Markup Language).

The trace discovery stage 128 extracts, in operation 230, difference traces in the event log(s) generated by the event log distiller 124. In one embodiment, the trace discovery stage may use an episode grouper to identify a set of relevant traces related to specific episodes of care. An example of an episode grouper is http://www.prometheusanalytics.net/deeper-dive/episode-care-definitions, provided by Prometheus Analytics. This episode grouper offers a description of episodes of care definitions which may be used as a blueprint to identify relevant traces in the event log(s). The traces may be associated with a number of parameters (e.g., patient, providers involved, conditions treated, etc.) that can be used to group the traces in the next steps.

The second processing path 130 includes a patient profile distiller 134 and a patient clustering stage 138. The patient profile distiller 134 may perform a role similar to the event log distiller 124 for purposes of generating, at operation 240, one or more event logs based on second information stored in the data manager module 110. In one embodiment, the second information may include patient profiles determined based on the information stored in the data manager module 110. The patient profiles may correspond, for example, to those patients that are of relevance, for example, ones that correspond to predetermined medical conditions, insurance participation, treatment options, and/or any other information that beforehand may be considered relevant for generating the patient profile event logs. In one embodiment, the set of patients corresponding to the patient profile event logs may be predetermined or extracted from results of the process performed by the trace discovery stage 128, as a trace may be linked to a patient. In this or another embodiment, the patient profile distiller 134 may extract key patient features (e.g., clinical status, demographics, and socio-economics) from the second information stored in the data manager module 110 to generate the patient profile event logs.

The patient clustering stage 138 determines, at operation 250, a plurality of clusters (or patient groups) based on the patient profile event log(s) output from the patient profile distiller 134. The patient clustering stage 138 performs this operation based on one or more clustering techniques that take into consideration features that allow similar patients to be identified at the start of an episode of care. The result of the patient clustering technique(s) is an output that corresponds to a limited set of patient clusters, where each cluster includes a (reasonably) homogeneous group of patients. In one embodiment, each cluster may group patients having at least one common feature relating to treatment, disease, condition, and/or one or more other parameters. The clustering performed by stage 138, therefore, allows the amount of variation typically present in healthcare-related processes to be efficiently and effectively managed, and allows processes followed by similar patients to be compared in a subsequent operation. Examples of clustering techniques include, but are not limited to, hierarchical agglomerative or divisive methods and latent class analysis methods.

Referring again to FIG. 1, the system 100 further includes a process grouping module 140, a processing mining module 150, a costing module 160, and a process analysis module 170. The process grouping module 140 receives the output of the first and second processing paths 120 and 130. More particularly, the process grouping module 140 receives the trace instances output from the trace discovery stage 128 and the patient clusters (or groups) output from the patient clustering stage 134. Once the traces are received, the process grouping module 140 groups, at operation 260, the traces to form sets of traces linked to the patient groups. The sets of traces linked to similar patients are input, one-by-one, into the process mining stage 150 at operation 270.

The process mining module 150 generates, at operation 310, a process model based on the sets of traces (which are linked to similar patients) output from the process grouping stage. Even though the process grouping stage 140 has already created trace sets belonging to similar patients, a reasonable amount of variation within each trace set may still be expected. Therefore, in accordance with one embodiment, a clustering technique may be applied to cluster the similar traces within the trace set. One example of a clustering techniques that may be used for this purpose includes sequence clustering, e.g., such as disclosed in “Developing Process Mining Tools, An Implementation of Sequence Clustering for ProM”, Master Thesis by Gabriel Martens Veiga, Technical University of Lisbon, 2009). The technique disclosed in this publication has been shown to produce simpler results than trace clustering, at least in some circumstances. This technique will also result in a Markov chain for each cluster, which can be easily visualized. The state space of the Markov chain may be given by different tasks in the event logs and a start state and an end state. The Markov chain may provide insight into what tasks can be executed next with a certain probability given some present task.

The costing module 160 may assign, at operation 320, a cost to each state of the Markov chain. This may be accomplished, for example, by estimating the cost of each state in the chain based on the resources used for it. According to one example, assume that each state Sᵢ in the Markov chains has been assigned a corresponding cost cᵢ. A Monte-Carlo simulation may be applied in order to compute an expected cost ecₚₗ for each chain Cₗ in the trace set Tₚ of a patient group (cluster) Pₚ. Furthermore, each trace tₖ from Tₚ may be assigned to the chain Cₗ with the highest probability of producing tₖ.

In one embodiment, at operation 330, weights may be calculated for each of the Markov chains. The weight wₗ of each chain Cₗ may be computed, for example, by dividing the number of traces assigned to chain Cₗ by the size of the trace set including the number of traces. If the sum of the weights of the chains for a trace set is assumed to equal 1, then the expected cost ecₚ of the trace set may then be given by Equation (1).

Σₗwₗ×ecₚₗ (1)

Similar to the weight of a Markov chain, a weight Wₚ for each patient group (Pₚ)-related trace set Tₚ may be computed, at operation 340, as the weighted sum in Equation (2).

$$a \times \frac{\vert {P}_{p} \vert}{\# patients} + b \times \frac{\vert {T}_{p} \vert}{\# traces} \qquad ( 2 )$$

where a+b=1. The values of a and b are set to reflect the relative importance of the number of patients versus the number of traces. In may be assumed that in the following discussion, purely for illustrative purposes and for the sake of simplicity, that b=1 so that Wₚ equals the percentage of all traces that are in Tₚ. As such, the overall expected cost of the episode of care may then be calculated based on Equation (3).

ΣₚWₚ×ecₚ (3)

In addition to the cost and weight, a measure may be calculated which represents a similarity between the Markov chains for a patient group and between patient groups. Similarity of the chains for a patient group may be determined, for example, based on the technique for calculating distance measures between Markov chains disclosed in the article, “Estimating the Parameters of Randomly Interleaved Markov Models”, by Daniel Gillblad, Rebecca Steinert and Diogo R. Ferreira, in 2009 IEEE International Conference on Data Mining Workshops.

Applying this technique, a distance measure dₗₘ may be calculated, at operation 350, which returns a value in [0,1], where 0 indicates a distance of zero between a respective pair of Markov chains Cₗ and Cₘ. In order to apply the distance measure D_{pq} to represent a similarity between two sets of Markov chains for two patient groups Pₚ and P_{q}, an average Markov chain may be computed for each set. The distance measure may then be applied using these average Markov chains. In order to compute the average Markov chain for a patient group Pₚ with a set of chains C₁ . . . C_{L} with state sets S₁ . . . S_{L}, a transition matrix M^{p} may be calculated based on Equation (4) with state set Sₚ=unique(S₁ ∪ . . . ∪ S_{L}) for 1≤i, j≤|Sₚ|:

$${M}_{{s}_{i} , {s}_{j}}^{p} = \frac{{\sum }_{\{ b : {s}_{i} ∈ {s}_{b} ⋀ {s}_{j} ∈ {s}_{b} \}} {w}_{b} \times {M}_{{s}_{i} , {s}_{j}}^{b}}{{\sum }_{\{ a : {s}_{i} ∈ {s}_{a} \}} {w}_{a}} \qquad ( 4 )$$

A description of the equations and variables that may be used to perform the techniques of the costing module 16 are given in the following table.

| Variable | Description | Method of construction/computation | Reference |
|---|---|---|---|
| Pₚ | homogenous patient | cluster algorithm, | |
| | group, patient cluster | selection based on patient profiles | |
| trace | sequence of | episode grouper, | Prometheus, |
| | associated health | bundled payments grouper, | CMS |
| | care events of one | source: claims data | |
| | patient, | | |
| | longitudinal patient | | |
| | trajectory | | |
| Tₚ | trace set of a patient | traces are augmented by other data sources | Described |
| | group Pₚ | and grouped together for each patient cluster | herein |
| Cₗ | Similar traces of a | sequence clustering for process mining | Developing |
| | trace set Tₚ are | | Process Mining |
| | clustered together. | | Tools, An |
| | Each cluster is | | Implementation |
| | associated with a | | of Sequence |
| | Markov chain Cₗ. | | Clustering for |
| | | | ProM |
| tₖ | Trace tₖ within trace | Pr(tₖ\|Cₗ) = p(s₁, Cₗ) · Πᵢ₌₂^{K}Pr(sᵢ\|sᵢ₋₁, Cₗ) | Described |
| | set Tₚ assigned to that | with trace sequence tₖ = (s₁, s₂, . . . , s_{K}) | herein |
| | Markov chain Cₗ | and tₖ → Cₗ for l with | |
| | with maximum | | |
| | probability of generating that trace | $Pr ( {t}_{k} \vert {C}_{l} ) = {max}_{l} Pr ( {t}_{k} \vert {C}_{l} )$ | |
| Sₗ = {sᵢ} | state space of the | states can comprise health care utilization | Described |
| | Markow chain Cₗ | events (e.g. ED visit, inpatient admission or | herein |
| | where each state sᵢ | other places of service delivery), diagnosis | |
| | represents a task in | related events or procedural events indicated | |
| | event log | e.g. by HCPCS or ICD codes or can related to | |
| | | visits to or services rendered by a provider | |
| | | with a given NPI | |
| cᵢ | cost assigned to each | It is given that each health care provider | TDABC method |
| | state sᵢ in the Markow | established an TDABC cost estimate for each | and Described |
| | chain Cₗ | sequence of an episode they render services | herein |
| | | for. | |
| | | If not actual cost information is available for | |
| | | an identified state, cost can be imputed (e.g. | |
| | | by cost-to-charge ratios) | |
| ecₚₗ | expected cost for | Markov chain Monte Carlo (MCMC) | Described |
| | each chain Cₗ in the | simulation based on cᵢ for each state | herein |
| | trace set Tₚ derived by | | |
| | micro-simulation | | |
| ecₚ | expected cost of the | ecₚ = Σₗwₗ × ecₚₗ | Described |
| | trace set Tₚ | | herein |
| wₗ | weight of each chain Cₗ given by number of | ${w}_{l} = \frac{\# {t}_{k}}{\vert {T}_{p} \vert} where {\sum }_{l} {w}_{l} = 1$ | Described herein |
| | assigned traces tₖ | | |
| | divided by size of | | |
| | trace set | | |
| TC | total expected cost of | TC = ΣₚWₚ × ecₚ | Described |
| | the episode of care | | herein |
| | for a pool of patient | | |
| | groups | | |
| Wₚ | weight for each patient group Pₚ related trace set Tₚ | ${W}_{p} = a \times \frac{\vert {P}_{p} \vert}{\# patients} + b \times \frac{\vert {T}_{p} \vert}{\# traces}$ | Described herein |
| | | with a + b = 1 | |
| dₗₘ | the divergence | dₗₘ = log Pr(tₖ\|Cₗ) − log Pr(tₖ\|Cₘ) | A Probabilistic |
| | distance measure is | | Distance |
| | the difference in log | | Measure for |
| | probabilities of an | | Hidden Markov |
| | observed trace | | Models |
| | conditioned on the 2 | | |
| | Markov chains Cl, Cm | | |
| | being compared | | |
| D_{pq} | similarity between | apply the divergence distance measure on the | Described |
| | two sets of Markov | two average Markov chains for the two | herein |
| | chains for two patient | patient groups being compared | |
| | groups Pₚ, P_{q} | | |
| (Mₛ_{ᵢ}_{,s}_{ⱼ}^{p}) | transition matrix for the average Markov chain for a patient group Pₚ with set of | ${M}_{{s}_{i} , {s}_{j}}^{p} = \frac{{\sum }_{\{ b \text{:} {s}_{i} ∈ {S}_{b} ⩓ {s}_{j} ∈ {S}_{b} \}} {w}_{b} \times {M}_{{s}_{i} , {s}_{j}}^{b}}{{\sum }_{\{ a \text{:} {s}_{i} ∈ {S}_{a} \}} {w}_{a}}$ | Described herein |
| | chains C₁ . . . C_{L} with | | |
| | state sets S₁ . . . S_{L} | for 1 ≤ i, j ≤ \|Sₚ\| | |
| | | with Sₚ = unique(S₁ ∪ . . . ∪ S_{L}) | |
| (Mₛ_{ᵢ}_{,s}_{ⱼ}^{b}) | transition matrix for | Mₛ_{ᵢ}_{,s}_{ⱼ}^{l} = Pr(sⱼ\|sᵢ, Cₗ) | Described |
| | Markov chain C_{b} | | herein |
| | within a patient | | |
| | group with state set | | |
| | S_{b} | | |

The process analysis module 170 performs one or more processes to interpret the results generated from the costing module 160. This interepretaion may place the results in a form that is more understandable for an end user. For example, the process analysis module 170 may output information indicative of trends, patterns, and/or other insights corresponds to the results of the costing module. In one embodiment, at operation 360, process analysis module 170 may generate information that answers one or more of the following questions based on the patient groups, their associated Markov chains, and their attendant costs, weights, and distances.
Which patient group has the highest overall cost (using weight and cost at patient group level)?
Which patient group has on average the most expensive process (using cost and weight at chain level)?
Which patient group has a lot of variation in processes (using weight at chain level)?
Which is the most common process (using weight at group and chain level)?
What is the most common process for a particular patient group (using weight at chain level)?
How different are the processes between patient groups (using distance at group level)?
How different are the processes for a patient group (using distance at chain level)?

In one embodiment, the process analysis module 170 may generate a visual representation of the Markov chains, the distances between them, and their weights and costs in order to help improve the level of understanding of an end user of the different processes.

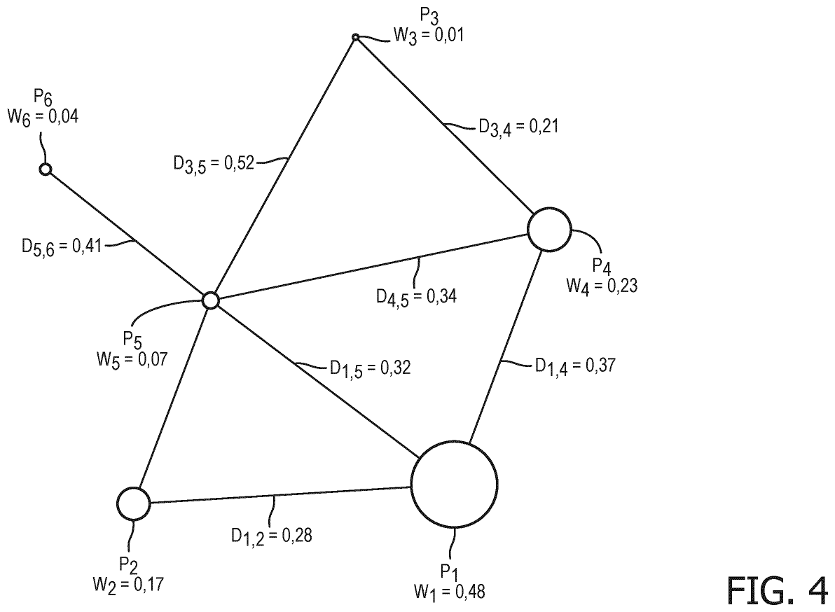

FIG. 4 illustrates an example of such a visual representation for the case of 6 patient groups (clusters) P1 to P6 with corresponding weights W₁ to W₆ and distance measures D_{a,b}, where a and b represent the Markov chains in each pair of a respective distance measure and the value of D represents the degree of similarity between the Markov chains/patient groups. The sizes of the circular spaces of each patient group may represent, for example, the number of processes performed. In this example, only edges with reasonably low distances (e.g., distances below a predetermined value) are depicted to keep the graph clean.

As illustrated in FIG. 4, the plot clearly shows that the patient group P1 has the highest weight. This indicates that patient group P1 has highest number of processes performed compared to the other five groups P2 to P6. Because the other groups have much lower weights, it may be determined that P1 represents a more typical patient group compared to the other patient groups.

Nonetheless, looking at the other groups may still important because, in this example, P2 and P4 also represent large numbers of executed processes. In addition, costs may be a helpful parameter, e.g., P6 may represent a rare variation but could be extremely expensive. Hence, it may interesting to understand the reason behind this high cost for this particular type of patient. Given similar information (e.g., weight, distances, cost, etc.) exists for each patient group, it is evident that a similar graph may be constructed and analyzed by the process analysis module 170 for each patient group individually. Also, the process analysis module 170 may provide a visual way to compare two (or more) process models based on their Markov chain representations. Comparing chains may be helpful, for example, in order to better understand where the cost difference is created between a common process that is relatively low in cost and a rare but very expensive process.

In another embodiment, the process analysis module 170 may estimate, at operation 360, profitability based on the average Markov chain per patient group. The profitability may be estimated, for example, for an episode budget contracted with a payer (e.g., insurer, health plan, TPA, employer) for delivering care for a defined patient pool. The contractual total episode budget (e.g. for a total knee replacement episode) may include the following elements in this example.

| Budget variable | Value |
|---|---|
| Budget for typical cost | $25,000 |
| Budget allowance for complications | $5,000 |
| Budget withhold (expected leakage) | $1,000 |
| Total episode budget (TB) | $29,000 |

In one embodiment, the process analysis module 170 may perform the profitability assessment based on the following algorithmic flow. First, each patient of the prospective patient pool with a bundled payment agreement may be assigned to a patient group Pₚ based on the minimal distance of the patient profile to that of the patient group.

Second, for each patient group Pₚ, only episodes for the contracted episode (e.g., total knee replacement) and its associated complication episodes are selected.

Third, an average Markov chain for the contracted episode is then computed for each of the patient groups.

Fourth, the total average expected episode cost for the prospective patient pool is computed based on Equation (5).

$$TC = \frac{1}{volume} {\sum }_{p} {W}_{p , contract} \times e {c}_{p , contract} \qquad ( 5 )$$

Fifth, if a decision on accepting or revising the contractual agreement is to be made, the decision may be made based on a computed expected margin using Equation (6).

$$m = \frac{TB - TC}{TB} \qquad ( 6 )$$

An example of the decision options based on Equation (6) is set for below.

| Decision | Decision Logic |
|---|---|
| Accept contract | Expected margin > threshold |
| Reject or revise contract | Expected margin ≤ threshold |

In this example, the expected average total cost was computed to be TC=$27,000 resulting in a margin of 6.8%. Under this contract, the health care organization (HCO) would still have room to accept a target on complication reduction. In a worst case scenario (e.g., where complications could not be reduced and hence TC would stay the same), the HCO could accept a reduction target of in average $2,000 with the potential gain in an incentive upon succeeded reduction in complications.

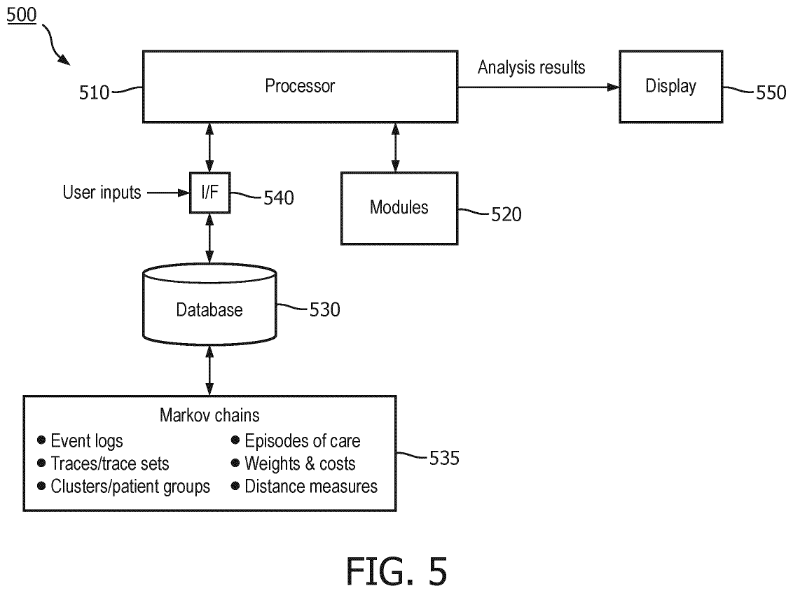

FIG. 5 illustrates an embodiment of a processing system 500 for managing the allocation and cost of healthcare resources. The processing system may include the modules, stages, and other features of FIG. 1 or may include one or more feature different from the system of FIG. 1.

Referring to FIG. 5, the processing system 500 includes a processor 510, a machine-readable storage medium 520, a database 530, an interface 540, and a display 550. The processor 510 may be implemented in logic which, for example, may include hardware, software, or both. When implemented at least partially in hardware, the processor 510 may be, for example, any one of a variety of integrated circuits including but not limited to an application-specific integrated circuit, a field-programmable gate array, a central processing unit, a combination of logic gates, a system-on-chip, a microprocessor, or another type of processing or control circuit.

When implemented in at least partially in software, the processor 510 may include or be coupled to a memory or other storage device (e.g., medium 520) for storing code or instructions to be executed, for example, by a computer, processor, microprocessor, controller, or other signal processing device. The code or instructions may include all or a portion of the modules, stages, and other features illustrated in FIG. 1. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the operations and methods of the embodiments described herein.

The machine-readable storage medium 520 stores instructions for controlling the processor 510 to perform some or all of the operations of the method and system embodiments described herein. In this case, the modules, stages, and/or other features (e.g., as set forth in FIG. 1) may be implemented in any of the forms of logic (software, hardware, or a combination) herein.

The database 530 stores various forms of information that may be generated and/or used by processor 510 to perform one or more of the aforementioned operations. In one embodiment, the database 530 may store the first information, second information, and/or other data in or derived from the data manager & input module 110 in FIG. 1. The database 530 may also store information corresponding to the first and second sets of event logs, traces, trace sets, patient groups, episodes of care, weights, distance measures, costs, and/or other features calculated, used, or stored by the embodiments described herein. The database 530 may also store the Markov chains and its associated data and information, as conceptually illustrated by block 535 in FIG. 5.

The database 530 may be or include a centralized database, a decentralized database (e.g., blockchain), or a storage network of databases respectively storing patient data, insurance claim data, socio-economic data, cost data, and/or other information used herein. In one embodiment, the database 530 may be at least partially implemented in a cloud-based network.

The interface 540 may be implemented in hardware, software, or both. When implemented in hardware, the interface 540 may include a port, connector, pin configuration, cable, or signal lines. In one embodiment, the interface may include a wireless interface (e.g., WiFi, GSM, CDMA, LTE, or other mobile network), or an interface compatible with another type of communication protocol). The interface 540 may transfer information between the processor 510 and the database 530, including but not limited to data genearted based on operations of the modules 520. The interface 540 may also receive information from a user to control the processor and modules, e.g., to update the processor or modules with new, different, or updated parameters, etc.

In one case, the processor 510 may be located remotely from the display 550, e.g., may be included in a virtual private network accessible by personnel at different locations. When implemented in software, an interface between the processor 510 and display 550 may include, for example, application programming interface (API) running on a workstation, server, client, or mobile device.

In operation, the instructions stored in the machine-readable medium 520 controls the processor 510 to perform the operations of the method and system embodiments described herein. The processor may receive inputs from one or more users, applications, and/or control software during this time to control, change, or implement some of these operations. The results of the processor 510, including a designation of profitability and/or allocation of healthcare resources and/or other insights and analysis results, for example, output from the process analysis module 170, may be displayed on the display 550.

### Technological Innovation

One or more embodiments described herein address a problem and/or provide a technical solution to managing healthcare services and expenditures in a way not previously known or practiced. For example, one problem in the field is assessing actual costs associated with various solutions for value-based care. Existing attempts have encountered at least the following problems:
Existing methods for developing process maps and time estimates are resource intensive, as they typically require techniques such as observations, surveys, or interviews.
Existing methods produce inaccurate results because of inconsistences in observations, surveys, and interviews. This may be exacerbated by bias and often the high variation that is associated with small numbers of observations (e.g., variation in physician capacity across specialties involved in a single care process). Further, it is difficult to capture accurate time estimates for activities that are seldom performed.
There is a lot of variation in the care processes due to the highly dynamic, complex, ad hoc, and multi-disciplinary nature of the processes.
Existing methods provide care in silo-ed departments/organizations. As a result, it is difficult to apply existing methods across a whole cycle of care stretching across organizational boundaries.
Existing methods do not provide accurate estimates of all costs involved, and do not take into consideration indirect costs.

One or more embodiments described herein solve these technological problems, by presenting solutions that are less resource intensive due to automation, more accurate in terms of results (e.g., no bias), more consistent in terms of the results provided, more complete in terms of processing a greater number of variations, and easier to maintain and update using an easily repeatable process. In addition, one or more embodiments described herein are efficient in terms of transferability and universality, and also are easily adaptive to other healthcare systems and organizations.

Additionally, while one or more features of the embodiments may involve the use of a mathematical formula, the embodiments are in no way restricted solely to a mathematical formula. Nor are they directed to a method of organizing human activity or a mental process. Rather, the complex and specific approach taken by the embodiments, combined with the amount of information processing performed, negate the possibility of the embodiments being performed by human activity or a mental process. Moreover, while a computer or other form of processor may be used to implement one or more features of the embodiments, the embodiments are not solely directed to using a computer as a tool to otherwise perform a process that was previously performed manually.

Nor do these embodiments preempt the general concept of making healthcare cost decisions. Rather, the embodiments disclosed herein take a specific approach (e.g., through event logs, trace sets, clustering algorithms, and weighting and distance measuring models) to solving technological problems that do not preempt, or otherwise restrict the public from practicing the general concept of, allocating healthcare resources.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

The modules, stages, models, processors, and other information generating, processing, and calculating features of the embodiments disclosed herein may be implemented in logic which, for example, may include hardware, software, or both. When implemented at least partially in hardware, the modules, models, engines, processors, and other information generating, processing, or calculating features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the modules, models, engines, processors, and other information generating, processing, or calculating features may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods herein.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

We claim:

1. A method for managing healthcare costs, comprising:
generating a first set of event logs based on first information;
extracting traces from the first set of event logs for different episodes of care;
generating a second set of event logs based on second information;
determining a plurality of clusters based on the second set of event logs;
grouping the traces to form sets of traces linked to the plurality of clusters; and
inputting the sets of traces into a process mining module.

2. The method of claim 1, wherein:
the first information includes patient or medical-care information;
the second information includes patient profile data, and
each of the plurality of clusters corresponds to different groups of patients.

3. The method of claim 2, wherein the patient profile data corresponds to at least one of medical conditions, insurance participation, or treatment options.

4. The method of claim 2, further comprising:
identifying patients corresponding to the patient profile data of the second set of event logs based on the traces extracted for the first set of event logs.

5. The method of claim 2, wherein patients in each patient group have at least one common feature.

6. The method of claim 2, further comprising:
generating, by the process mining module, a Markov chain for each cluster in the set of clusters, wherein a state space of the Markov chain of each cluster is given by different tasks indicated in the event logs of the first set.

7. The method of claim 6, further comprising:
assigning a cost to states in one or more of the Markov chains; and
calculating weights for the Markov chains based on a ratio of a number of the traces assigned to each of the Markov chains to a size of the trace set including the number of traces.

8. The method of claim 7, further comprising:
calculating distance measures for respective pairs of the Markov chains,
wherein each of the distance measures provides an indication of similarity between the Markov chains in a respective one of the pairs.

9. The method of claim 8, further comprising:
determining an expected cost of each of the different episodes of care for each patient group based on the calculated costs, weights, and distance measures.

10. The method of claim 9, further comprising:
estimating profitability for each of the different episodes of care for each patient group, the profitability estimated based on a budget for each different episode contracted with a payer less the expected cost of the different episode.

11. A system for managing healthcare costs, comprising:
an interface to receive first information and second information; and
a processor configured to generate a first set of event logs based on the first information, extract traces from the first set of event logs for different episodes of care, generate a second set of event logs based on the second information, determine a plurality of clusters based on the second set of event logs, group the traces to form sets of traces linked to the plurality of clusters, input the sets of traces into a process mining module, and output results based on the process mining module for display.

12. The system of claim 11 wherein:
the first information includes patient or medical-care information;
the second information includes patient profile data, and
each of the plurality of clusters corresponds to different groups of patients.

13. The system of claim 12, wherein the patient profile data corresponds to at least one of medical conditions, insurance participation, or treatment options.

14. The system of claim 12, wherein the processor is configured to identify patients corresponding to the patient profile data of the second set of event logs based on the traces extracted for the first set of event logs.

15. The system of claim 12, wherein patients in each patient group have at least one common feature.

16. The system of claim 12, wherein the processor is configured to generate a Markov chain for each cluster in the set of clusters, wherein a state space of the Markov chain of each cluster is given by different tasks indicated in the event logs of the first set.

17. The system of claim 16, wherein the processor is configured to assign a cost to states in one or more of the Markov chains.

18. The system of claim 17, wherein the processor is configured to calculate weights for the Markov chains based on a ratio of a number of the traces assigned to each of the Markov chains to a size of the trace set including the number of traces.

19. The system of claim 18, wherein the processor is configured to calculate distance measures for respective pairs of the Markov chains, wherein each of the distance measures provides an indication of similarity between the Markov chains in a respective one of the pairs.

20. The system of claim 19, wherein the processor is configured to determine an expected cost of each of the different episodes of care for each patient group based on the calculated costs, weights, and distance measures.
